(19) United States
(12) Patent Application Publication
(10) Pub. No.: US 2023/0381160 A1
(43) Pub. Date: Nov. 30, 2023

(54) PROPHYLATIC USE OF CALCIUM CHANNEL BLOCKERS IN TUBERCULOSIS

(71) Applicants: Chien-Chang LEE, Taipei City (TW); Matthew LEE, Taipei City (TW)

(72) Inventors: Chien-Chang LEE, Taipei City (TW); Matthew LEE, Taipei City (TW)

(21) Appl. No.: 18/028,233

(22) PCT Filed: Sep. 24, 2021
(86) PCT No.: PCT/US2021/051850

§ 371 Date: Mar. 24, 2023

Related U.S. Application Data
(60) Provisional application No. 63/082,476, filed on Sep. 24, 2020.

(51) Int. Cl. A61K 31/4422; A61P 31/06; G16H 70/40

(52) U.S. Cl. CPC A61K 31/4422; A61P 31/06; G16H 70/40

(57) ABSTRACT

This invention discloses a method of using calcium channel blockers as a prophylaxis in a predetermined population susceptible to tuberculosis. Also disclosed herein are pharmaceutical compositions useful for preventing or reducing the risk of activating latent TB in a patient infected with HIV, and a computer-aided drug development method for developing and optimizing a pharmaceutical composition useful for preventing or reducing the risk of activating latent TB.

### FIELD OF THE INVENTION

This invention discloses a method of using calcium channel blocker in a prophylactic capacity in a patient population, details of which are substantially as described in the attached manuscript.

### BACKGROUND OF THE INVENTION

Despite nearly two decades of worldwide anti-tuberculosis treatment programs, tuberculosis (TB) still remains the leading cause of death in the world. According to the World Health Organization (WHO), approximately 9 million people developed active TB and 1.5 million people died from the associated complications in 2013. Current therapy against TB, although effective, requires 6 to 9 months of treatment and is associated with drug toxicity. In addition, the antibiotic-based treatment strategies may foster multidrug resistant TB. More importantly, for AIDS patients, the chance of latent TB becoming active is much higher than patients without AIDS. Once these immune-compromised patients become active TB cases, there is currently no treatment for them and their most likely outcome is death. Therefore, there is a need for preventive strategy against TB.

### SUMMARY OF THE INVENTION

We disclose herein our surprising discoveries that (1) chronic use of dihydropyridine CCB may lower the risk of developing TB in general population; (2) dihydropyridine CCBs has a stronger protective effect than non-dihydropyridine CCBs, β-blockers and loop diuretics, and (3) dihydropyridine CCBs may be the preferred treatment for hypertension patients with TB.

In accordance with the surprising discoveries disclosed herein, the present invention provides methods for reducing the risk of latent TB activation and pharmaceutical compositions useful as prophylactics for preventing latent TB activation.

Accordingly, in one aspect, the present invention provides a preventive method of reducing the risk of activating latent TB in a subject in need of the preventive treatment by administering a calcium channel blocker on a regular basis prior to activation of latent TB.

Calcium channel blocker suitable for use in the method may be any known or future discovered calcium channel blocker, including but not limited to verapamil, nifedipine, and diltiazem. In a preferred embodiment, calcium channel blocker is a dihydropyridine calcium channel blocker, more preferably one previously approved by the FDA. In a more preferred embodiment, the calcium channel blocker is nifedipine.

Subjects who are in need of the preventive treatment are anyone who is at risk of exposure to TB or already been infected with TB but in a latent state of infection. More preferably, the subject is one who is also infected with HIV.

In another aspect, the present invention also provides a pharmaceutical composition useful as a prophylactic to reduce the risk of activating latent TB in a subject. Compositions in accordance with aspect of the present invention generally include a calcium channel blocker in combination with an anti-HIV agent, and a pharmaceutically acceptable carrier. Suitable calcium channel blockers are as described above. Suitable anti-HIV agents may be any anti-HIV agents known in the art, including but not limited to abacavir, emtricitabine, lamivudine, tenofovir disoproxil fumarate, zidovudine, doravirine, efavirenz, etravirenz, etravirine, nevirapine, ripivirine, atazanavir, darunavir, fosamprenavir, ritonavir, saquinavir, tipranavir, enfuvirtide, maraviroc, cabotegravir, dolutegravir, raltegravir, fostemsavir, ibalizumab-uiyk, cobicistat, or a combination thereof. In a preferred embodiment, both the calcium channel blocker and the anti-HIV agent are previously approved drugs by the FDA.

In yet another aspect, the present invention also provides a method for developing and optimizing a pharmaceutical composition suitable for reducing the risk of activating latent TB in a subject suffering from HIV. Methods in accordance with this aspect of the invention will generally include the steps of compiling a structural database of calcium channel blockers and a structural database of anti-HIV agents; ranking the calcium channel blockers and the anti-HIV agents according to their effectiveness; generating a pharmacophore model for each of calcium channel blocker and anti-HIV agent based on the top 10 ranked structures of each and performing a structural search on a database of potential drugs not including the calcium channel blockers and the anti-HIV agents using the pharmacophores.

The following example further describes the details of our discoveries.

### BRIEF DESCRIPTION OF THE DRAWINGS

FIG. 1 shows a delineates the patient inclusion and exclusion criteria.

FIG. 2 shows a summary of subgroup analysis.

### DETAILED DESCRIPTION

### Example

### Theoretical Background

Iron acquisition is essential for the growth of several intracellular pathogens, including Mycobacterium tuberculosis, promoting its infection, replication, and progression to clinical disease and death. The relationship of iron availability to mycobacterial growth in serum, cells, and mice has been demonstrated by several experimental studies. In humans, patients with TB often develop anemia of chronic disease (ACD), an inflammatory response resulting in iron dysregulation, where the body sequesters iron to prevent use by organisms such as the M. tuberculosis. Therefore, patients with TB may have a high ferritin level and a corresponding low serum iron and transferrin level. It was observed in an African cohort that high ferritin levels (plasma ferritin>150 μg/L for women and >200 mg/L for men) were seen in 48% of patients with newly diagnosed TB.

While not intending to be bound by any particular theory, we hypothesize that deprivation of iron may reduce M. tuberculosis viability and replication, and this concept may be applicable to preventing latent TB reactivation. In vitro studies showed that L-type voltage-gated calcium channels provided an alternate pathway for iron entry into different types of cells. Calcium channel blockers (CCBs) including verapamil, nifedipine, and diltiazem are directed against L-type voltage-gated Ca²⁺ channel, and several studies found that the use of CCBs can decrease the plasma iron level. Recent human trials also showed that treatment of thalassemia patients with the CCB amlodipine significantly reduced cardiac iron loading compared with placebo.

### Study Database

National Health Insurance (NHI) program of Taiwan is a compulsory single-payer system that enrolls approximately 24 million residents with 99.8% coverage. We conducted a nested case-control study using the National Health Insurance Research Database (NHIRD) approved from the institutional review board of National Taiwan University Hospital. The NHIRD used a systematic approach to randomly select one million people from the enrollees of NHI, which represents the age, sex, and geographical region distribution of the Taiwanese population. The computerized NHIRD includes information on patient demographics, outpatient and inpatient electronic claim records, individual diagnoses by ICD-9 CM codes, operations, and prescribed medications. Detailed information regarding the name of the prescribed drugs, route of administration, quantity, and number of days of supply are also available. The study was approved by the Institutional Review Board at National Taiwan University Hospital (No. 201311043RINB). Patients' consent was waived because this is an electronic database study using anonymous subjects.

### Study Cohort

The study cohort consisted of all adult patients (≥18 years old) from the NHIRD that were followed longitudinally between January 1999 and December 2011. We used 1999 as pre-enrollment periods. TB was defined as those with any code for TB according to the ICD-9 CM codes (ICD-9 CM codes: 010-018, including all subcategories). All participants in the study cohort were followed and contributed person-time from the first day of 2000 until the earliest of the following end points: first diagnosis of active TB, termination of health insurance coverage, death, or the end of the study period, whichever came first. The year before the year of TB diagnosis was used for the evaluation of the drug exposure.

### Ascertainment of Active TB

We identified incident cases of active TB by the following criteria: presence of ICD-9-CM codes of TB (010-018, including all subcategories) in at least one outpatient visit or one hospital admission record, plus the prescription of more than two anti-tuberculosis medications (i.e., isoniazid, rifampin, pyrazinamide, ethambutol, streptomycin, cycloserine, prothionamide, amikacin, or kanamycin) for more than 28 days. The case was excluded if patients had a subsequent diagnosis of non-tuberculosis mycobacterial infection or lung cancer. This set of TB definitions has been validated in previous studies. The date of TB onset (index date) was defined as the date of the first diagnosis of TB identified by the criteria above. Controls were assigned the same index date as the cases, ensuring that both cases and matched controls had an equal duration of follow-up before the index date.

### Selection of Controls

We selected controls by using a risk set sampling scheme. We sampled 100 controls for each incident case of active TB matched on 5-year age category and sex. We applied the same exclusion criteria to controls as to cases.

### Medication Exposure

The one-year period preceding the index date of TB diagnosis in cases and matched controls was used for assessment of all medications of interest. The CCBs were classified into two categories. Dihydropyridine CCBs refer to drugs including nifedipine, isradipine, nicardipine, felodipine, and amlodipine. Non-dihydropyridine CCBs refer to drugs including verapamil or diltiazem. The use of CCBs in the previous year was defined by having a reimbursement code for CCB with a cumulative prescription length for 60 days. To account for the recency of the CCB use, current use was defined if there was a supply of the most recent prescription that covered the index date or had ended within 90 days of the index date. The reference category for all analyses consisted of non-CCB use.

### Covariates

We assessed the covariates from the cohort entry to the year before the index date. Covariates assessed included: demographics, pre-existing chronic diseases, medications used, and proxy indicators for the lifestyle such as alcohol consumption or smoking related illness, and obesity or malnutrition. We also ascertained the numbers of visits to an outpatient clinic, emergency department, and hospital admission in the year before the index date. We used a combined comorbidity index that combined the Charlson Index with the Elixhauser system to quantify the total burden of chronic diseases for each individual. The full list of covariates is listed in Table S1.

### Data Analysis

Continuous variables were presented as means±standard deviation, and categorical variables were presented as frequency and percentage. Multigroup comparisons of continuous variables were performed using 1-way analysis of variance (ANOVA) tests, and multigroup categorical variable comparisons were conducted using chi-square tests. We estimated the odd ratios (ORs) of active TB (plus 95% confidence intervals [CIs]) using conditional logistic regression. Under a time-matched case-control sampling scheme, the odds ratio is an unbiased estimator of the incidence rate ratio, and we report odds ratio estimates as relative risks throughout this disclosure.

### Multivariate Analysis

We constructed two models to analyze the association between the use of CCBs and risk of active TB. The first model was a conditional logistic regression analysis that adjusted for all covariates except for the three matching variables (year of TB diagnosis, age, and gender). In the second model, we used a study-specific disease risk score (DRS) for adjustment using the approach proposed by Miettinen. The DRS was defined as the probability of developing incident active TB in a given year among all members of the cohort unexposed to CCBs as a function of individuals' covariates. We created the DRS in the source population by fitting a multivariate logistic regression model. We entered the DRS into the logistic regression model with a main term and a quadratic term to allow a nonlinear association between the DRS and the log odds of incident active TB.

To further assess the robustness of our results we performed a duration-response analysis, dose-response analysis, active comparator analysis, and sensitivity analysis. For the duration-response analysis, we categorized the duration of CCB use into three categories (light use 7-30 days, moderate use 31-90 days, and heavy use 91-365 days) and calculated the incidence and relative risk of active TB for each category. Then, we tested the linear association between the drug use duration and the risk of active TB. Since we had sufficient numbers of patients receiving a daily dose of 20 mg nifedipine or 30 mg nifedipine, we further conducted a dose-response analysis in these two groups. To examine whether unmeasured confounders such as healthy seeking behaviors played a role in the observed association, we conducted an active comparator analysis. We used beta-adrenergic blockers and loop diuretics as active comparators, and tested the association between use of beta-blockers and active TB as well as the association between use of loop diuretics and active TB. In the sensitivity analysis, we evaluated the impact of a missing confounder by the rule-out approach proposed by Schneeweiss. The analysis estimates the strength of the hypothetical binary confounder that is required to account for the observed effect of CCB.

Lastly, we performed a subgroup analysis in predefined subgroups. Predefined subgroups included age, sex, area of stay, insurance premium, and presence of comorbidity as defined by previously validated ICD-9 codes, used in the determination of combined comorbidity index.²³ All the analyses were performed with SAS 9.3 for Windows (SAS Institute Inc, Cary, NC).

### Patient Involvement

No patients were involved in setting the research question or the outcome measures, and they were also not involved in developing plans for design or implementation of the study. There are no plans to involve patients in dissemination.

### Results

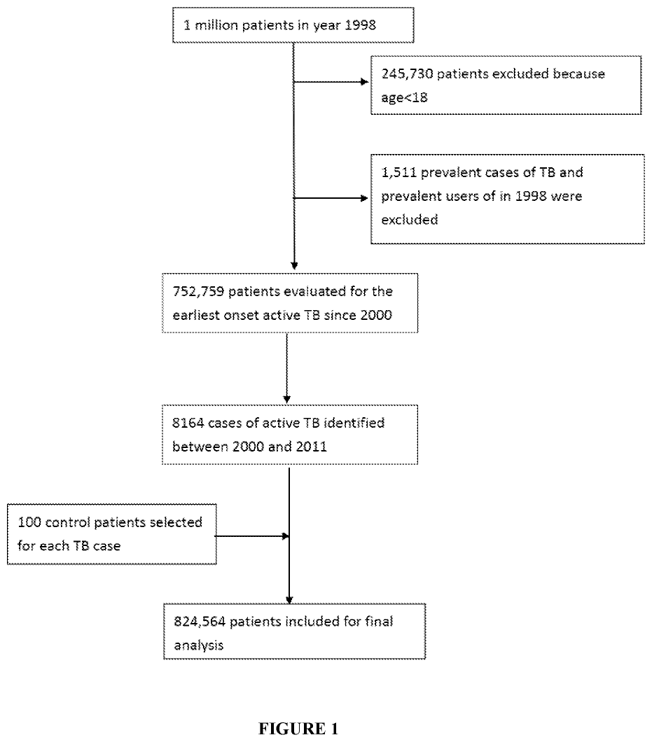

FIG. 1 delineates the patient inclusion and exclusion criteria. The final cohort encompassed 824,564 individuals followed for 6.37 years on average. Table S2 summarizes the baseline clinical characteristics of 8164 TB cases and 816,400 controls.

Table 1 compared the baseline among users of dihydropyridine CCBs, non-dihydropyridine CCBs, and non-users in the control population. There were 36,643 non-dihydropyridine CCB users, 69,205 dihydropyridine CCB users, and 717,473 participants unexposed to both types of CCBs. Compared with nonusers, users of either type of CCBs were observed to be older, more likely to pay a low insurance premium, and live in the suburbs or rural areas. They were also more likely to have a higher burden of comorbidities, more risk factors for TB, higher frequency of healthcare facility utilization, and take more co-medications. Users of both types of CCBs had similar demographic profiles, but users of non-dihydropyridine CCBs had a higher burden of cardiac and pulmonary comorbidities and used health care services more frequently.

Relative Risk of Incident TB Associated with CCBs

Table 2 shows adjusted estimates of the effects of current exposures to different types of CCBs on new onset active TB from the conditional logistic regression analysis of cases and controls after matching for age, gender, and year. The adjusted relative risk associating current use of any CCBs with new onset of active TB was 0.77 (95% CI: 0.65-0.90). Adjustment for confounders decreased the relative risk to 0.67 (95% CI: 0.54-0.79). Adjustment for the DRS yielded similar effect estimates (0.68, 95% CI: 0.58-0.78). Using non-CCB use as the reference group, dihydropyridine CCBs were associated with lower risk for onset of active TB (0.63, 95% CI: 0.53-0.79) than non-dihydropyridine CCBs (0.73, 95% CI: 0.57-0.94). The c-statistic of the DRS model was 0.83. The component variables of the DRS model and their corresponding weights are shown in the Table S1.

### Duration and Dose Analysis

The incidence of active TB decreased as the duration of CCBs use increased. Multivariate analysis showed that the risk of incident active TB decreased from light- to moderate- to heavy-use categories (trend P<0.0001). A daily dose of 20 mg Nifedipine or 30 mg Nifedipine were both associated with a decreased risk of active TB, but the level of protection was unrelated to the dose (trend P<0.44) (Table 3).

### Active Comparator Analysis

To gain insights into whether the observed association could be explained by healthy user bias, we compared the risk of active TB between CCB and two other cardiovascular medications (beta-blockers and loop diuretics) (Table 4). The use of CCB was associated with a decreased risk of active TB after individual confounder adjustment (RR 0.67, 95% CI: 0.54-0.79), and DRS score adjustment (RR, 0.68, 95% CI: 0.58-0.78), as compared with nonuse. In contrast, the use of beta-blockers, an active comparator, was not associated with a decreased risk of active TB after individual confounder adjustment (RR 0.92, 95% CI: 0.81-1.06), and DRS score adjustment (RR, 0.99, 95% CI: 0.83-1.12), when compared with nonuse. Similar results were obtained, when loop diuretics were used as an active comparator. Use of loop diuretics were not associated with a decreased risk of active TB.

### Sensitivity Analysis

We conducted the rule-out approach sensitivity analysis to estimate the strength of hypothetical confounders to explain the TB protective effect of CCBs (DRS-adjusted RR=0.66 or 0.9). Assuming an unmeasured confounder present at 20% of the population and the prevalence of CCB exposure is 1%, the analysis shows that exposure-confounder associations able to move the estimated OR to the null when confounder-disease associations were approximately at 4 and 7, which is highly unlikely.

### Patient Subgroup Analyses

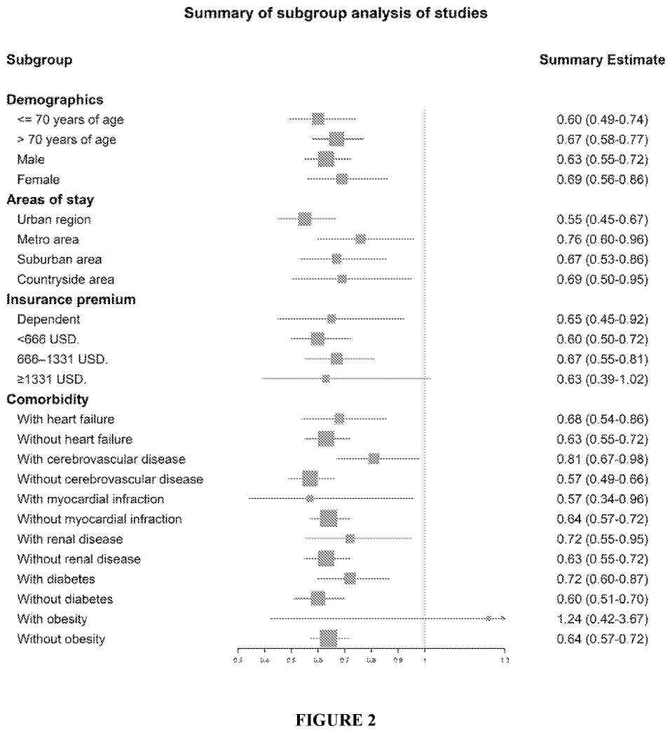

The subgroup analyses adjusted by DRS showed that the risk of TB associated with the use of CCBs were very similar across all subgroups (elderly age, gender, heart failure, cerebrovascular diseases, renal diseases and diabetes). The only exception was in the obesity subgroup, however, the small size resulted in large confidence intervals (FIG. 2).

### Discussion

In this large population-based cohort study, we found that users of CCBs experienced a decreased risk of active TB compared with CCB nonusers. The decreased risk estimate persisted after comprehensive adjustment for potential confounders. The relative risk decrease was higher among users of dihydropyridine CCBs in comparison to users of non-dihydropyridine CCBs. Longer use of CCBs prior to TB onset was associated with lower risk of incident active TB. We did not find that age, gender, or presence of cardiovascular comorbidity, renal disease, or diabetes modified the effect of CCBs on active TB. This effect of CCBs was partially highlighted by a previous study by Lee et al. (Lee M-Y, Lin K-D, Hsu W-H, et al. Statin, calcium channel blocker and Beta blocker therapy may decrease the incidence of tuberculosis infection in elderly Taiwanese patients with type 2 diabetes. International journal of molecular sciences. 2015; 16(5):11369-11384, the entire content of which is incorporated herein by reference) which demonstrated that CCBs, beta blockers and statin therapy decreased the incidence of TB infection in elderly Taiwanese patients with type 2 diabetes.

Our study design does not permit mechanistic insights on the differential TB protective effects of the dihydropyridine and non-dihydropyridine CCBs. However, our results agree with current experimental studies. The dihydropyridine and non-dihydropyridine CCBs differ not only in their basic chemical structure, but also in their relative selectivity toward vascular versus cardiac L-type calcium channels. In general, dihydropyridine CCBs such as nifedipine, have been found to be more selective towards vascular channels, while the non-dihydropyridine CCBs such as Verapamil (phenylalkylamine class), have been found to be more selective for the cardiac channels. Since dihydropyridine CCBs might have a more systemic effect, we speculate that dihydropyridine CCBs may be more effective in mobilizing iron. To the best of our knowledge, only nifedipine was reported to increase iron cellular transport in human embryonic kidney cells, and only nifedipine was found to reduce iron availability for intra-macrophage pathogens and to improve survival in animals infected with Salmonella or other intracellular pathogens. Iron is an essential nutrient for pathogens residing within macrophages. Iron accumulation may increase the risk for outgrowth of intracellular pathogens like Mycobacterium tuberculosis and inhibits the antimicrobial immune effector pathways of macrophages. In human studies, several lines of evidence suggest that macrophage iron content is associated with the risk of developing active TB. Iron dysregulation has been associated with progression to TB among HIV-infected patients or household contacts. A study by Clark et al. theorized the use of CCBs to enhance the host immune system and alter pathogen life cycles and antibiotic tolerances, by modulating calcium dependent regulatory pathways.

The major strength of this study is a large number of TB cases with extensive longitudinal follow-up. Overall, we identified 8,164 new TB cases, which provide sufficient statistical power for the main effect as well as sensitivity analyses. The large sample size also allows us to investigate the class effect of CCB on TB. Despite rigorous statistical methods and including a broad range of potential confounders, an observational study performed in a claim database can never exclude the possibility of different types of biases such as confounding bias, protopathic bias, or misclassification bias. We used a disease-specific DRS to address this issue to minimize the possible confounding effect. The DRS has several advantages. Conventional individual confounder adjustment in a case-control study only collects covariate information in the relatively small case-control sample with a limited number of common covariates. In contrast, DRS can be built in the source population with a large sample size and rich covariate information. It has the ability of high predictability to balance disease risks among CCB users and non-users, and it can be used for balancing disease risks among multiple drug exposure groups independent of the changing indications for CCBs. In addition, we performed a sensitivity analysis to show that very strong risk factors for TB must be unmeasured and uncontrolled to explain the observed association, which is very unlikely. The potential for protopathic bias, interpreted as CCBs used for the treatment of early symptoms of TB before TB diagnosis, is minimal in this study. Hypertension or angina is rarely the early manifestation of TB, and we only studied the long-term users rather than recent initiators in our study. Lastly, it must be accounted for the difference between our results and Lee et al, regarding the relationship between beta blockers and TB. This discrepancy could either be related to possible differences in the two studies' populations or possible clinical confounders (such as body mass index, duration of diabetes, smoking and alcohol consumption) not taken into consideration by Lee et al. (supra).

Our study should be interpreted in the context of the limitations inherent in its design. First, we cannot exclude misclassification bias as microbiology data for TB is not available in this database. However, we did not merely rely on ICD-9 codes for TB diagnosis. Instead, we used a set of validated criteria that require ICD-9 codes as well as concomitant use of anti-TB medication for at least two months. To further improve the accuracy of TB diagnosis, we excluded subsequent diagnosis of NTM or lung cancer. Second, this study investigates mainly Taiwanese with low HIV/TB co-infection, while a previous study found that approximately 0.2% of TB patients in Taiwan have HIV/TB co-infection. When replicating our study in an African nation where the TB incidence is high and the HIV/TB co-infection rate is enormous, researchers may need to pay attention to the potential drug-drug interactions of CCBs on antiretroviral therapy. Previous pharmacokinetic studies showed increased serum level of CCBs when co-administered with indinavir and ritonavir. Our unique finding may have a significant impact on the TB prescribing habits of clinicians, especially in geographies such as Southern Africa where the rate of dual pathologies (HIV and hypertension) are high. Later real-life studies, however, showed such drug-drug interactions can be adequately managed by adjusting CCB doses with clinically monitoring blood pressure and electrocardiography. There is no need for discontinuation of the CCBs or change in antiretroviral regimen. In countries where diabetes mellitus (DM) is a major risk factor for TB, CCBs can also be a preferred antihypertensive drug as they have been linked with lower fasting blood glucose levels in a large prospective cohort with diabetes.

TABLE 1

| | Control group (N = 816,400 person-years) | | |
|---|---|---|---|
| | Non- | | |
| | dihydropyridine | Dihydropyridine | |
| | CCBs | CCB | Non-users |
| Variables | N = 36,643 | N = 69,200 | N = 717,473 |

Demographics

| | | | | |
|---|---|---|---|---|
| Male sex (%) | 26,054 (71.10) | 47,779 (69.04) | 491,584 (68.52) | <0.001 |
| Age mean (year) | 73.99 ± 10.40 | 72.62 ± 10.84 | 58.64 ± 19.37 | <0.001 |

Index year (%)

| | | | | |
|---|---|---|---|---|
| 1999 | 2205 (6.02) | 4325 (6.25) | 58607 (8.17) | <0.001 |
| 2000 | 3166 (8.64) | 5749 (8.31) | 59550 (8.30) | |
| 2001 | 3282 (8.96) | 5487 (7.93) | 57062 (7.95) | |
| 2002 | 3611 (9.85) | 5325 (7.70) | 63110 (8.80) | |
| 2003 | 3447 (9.41) | 5161 (7.46) | 60791 (8.47) | |
| 2004 | 3501 (9.55) | 5675 (8.20) | 64659 (9.01) | |
| 2005 | 3540 (9.66) | 6294 (9.10) | 64941 (9.05) | |
| 2006 | 2870 (7.83) | 5410 (7.82) | 54315 (7.57) | |
| 2007 | 2484 (6.78) | 5054 (7.30) | 49823 (6.94) | |
| 2008 | 2559 (6.98) | 5690 (8.22) | 52605 (7.33) | |
| 2009 | 2239 (6.11) | 5354 (7.74) | 46293 (6.45) | |
| 2010 | 2025 (5.53) | 5233 (7.56) | 45482 (6.34) | |
| 2011 | 1714 (4.68) | 4443 (6.42) | 40235 (5.61) | |
| Area of stay | | | | |
| Urban region | 14417 (39.34) | 27077 (39.13) | 349424 (48.70) | <0.001 |
| Metro area | 9279 (25.32) | 17713 (25.60) | 178552 (24.89) | |
| Suburban area | 8697 (23.73) | 16002 (23.12) | 132415 (18.46) | |
| Countryside area | 4250 (11.60) | 8408 (12.15) | 57082 (7.96) | |

Insurance premiums

| Dependent | | 5171 (14.11) | 9235 (13.35) | 64883 (9.04) | <0.001 |
|---|---|---|---|---|---|
| <666 | USD | 15627 (42.65) | 27285 (39.43) | 201801 (28.13) | |
| 666-1331 | USD | 12596 (34.37) | 25577 (36.96) | 300492 (41.88) | |
| ≥1331 | USD | 3249 (8.87) | 7103 (10.26) | 150297 (20.95) | |

Comorbidity score

| | | | |
|---|---|---|---|
| Baseline | 2.38 ± 2.41 | 1.80 ± 2.24 | 0.96 ± 1.64 |
| combined | | | |
| comorbidity | | | |
| score | | | |

Conditions included in the Charlson index

| | | | | |
|---|---|---|---|---|
| Peripheral | 5155 (14.07) | 8786 (12.70) | 34021 (4.74) | <0.001 |
| vascular | | | | |
| disease | | | | |
| Congestive | 10847 (29.60) | 14829 (21.43) | 49245 (6.86) | <0.001 |
| heart failure | | | | |
| Myocardial | 3463 (9.45) | 3539 (5.11) | 13372 (1.86) | <0.001 |
| infarction/ | | | | |
| acute coronary | | | | |
| syndromes | | | | |
| Cerebrovascular | 13350 (36.43) | 24612 (35.57) | 98836 (13.78) | <0.001 |
| disease | | | | |
| Dementia | 2525 (6.89) | 4481 (6.48) | 22870 (3.19) | <0.001 |
| Chronic | 22386 (61.09) | 34520 (49.88) | 212366 (29.60) | <0.001 |
| pulmonary | | | | |
| disease | | | | |
| Rheumatologic | 1627 (4.44) | 2511 (3.63) | 16065 (2.24) | <0.001 |
| disease | | | | |
| Peptic ulcer | 18964 (51.75) | 32314 (46.70) | 208667 (29.08) | <0.001 |
| disease | | | | |
| Mild liver | 13229 (36.10) | 24011 (34.70) | 163237 (22.75) | <0.001 |
| disease | | | | |
| Moderate or | 221 (0.60) | 364 (0.53) | 3044 (0.42) | <0.001 |
| severe liver | | | | |
| disease | | | | |
| Diabetes | 12151 (33.16) | 24578 (35.52) | 108412 (15.11) | <0.001 |
| without chronic | | | | |
| complications | | | | |
| Diabetes with | 4349 (11.87) | 8975 (12.97) | 32060 (4.47) | <0.001 |
| chronic | | | | |
| complications | | | | |
| Hemiplegia | 2252 (6.15) | 4712 (6.81) | 21854 (3.05) | <0.001 |
| or paraplegia | | | | |
| Renal disease | 5636 (15.38) | 10505 (15.18) | 41975 (5.85) | <0.001 |
| Any | 5221 (14.25) | 8006 (11.57) | 50195 (7.00) | <0.001 |
| malignancy, | | | | |
| including | | | | |
| leukemia and | | | | |
| lymphoma | | | | |
| Metastatic | 487 (1.33) | 789 (1.14) | 5300 (0.74) | <0.001 |
| solid tumor | | | | |
| AIDS/HIV | 26 (0.07) | 22 (0.03) | 366 (0.05) | 0.01 |

Additional comorbidities

| | | | | |
|---|---|---|---|---|
| COPD | 18578 (50.70) | 27117 (39.19) | 148275 (20.67) | <0.001 |
| Silicosis | 73 (0.20) | 109 (0.16) | 677 (0.09) | <0.001 |
| Psychiatric | 17302 (47.22) | 27652 (39.96) | 167370 (23.33) | <0.001 |
| disorder | | | | |
| Neurologic | 3541 (9.66) | 5608 (8.10) | 30250 (4.22) | <0.001 |
| disorder | | | | |
| Gastrointestinal | 3219 (8.78) | 5540 (8.01) | 30459 (4.25) | <0.001 |
| or esophageal | | | | |
| hemorrhage | | | | |
| Malnutrition | 1668 (4.55) | 2527 (3.65) | 16028 (2.23) | <0.001 |
| Alcohol/drug | 1190 (3.25) | 1860 (2.69) | 14770 (2.06) | <0.001 |
| use | | | | |
| Obesity | 336 (0.92) | 709 (1.02) | 4573 (0.64) | <0.001 |
| Postgastric | 77 (0.21) | 87 (0.13) | 581 (0.08) | <0.001 |
| surgery | | | | |
| Solid organ | 23 (0.06) | 26 (0.04) | 130 (0.02) | <0.001 |
| transplantation | | | | |

OPD and hospitalization (within one year before the index date)

| | | | | |
|---|---|---|---|---|
| The number | 39.83 ± 26.64 | 35.88 ± 25.62 | 18.31 ± 19.47 | <0.001 |
| of OPD visit | | | | |
| The number | 0.33 ± 1.21 | 0.25 ± 1.04 | 0.12 ± 0.58 | <0.001 |
| of emergency | | | | |
| department | | | | |
| visit | | | | |
| The number of | 0.53 ± 1.17 | 0.40 ± 1.01 | 0.19 ± 0.94 | <0.001 |
| hospitalization | | | | |

Medication use

| | | | | |
|---|---|---|---|---|
| NSAID | 18.327 (50.02) | 33,504 (48.42) | 209,087 (29.14) | <0.001 |
| (Nonsteroidal | | | | |
| anti- | | | | |
| inflammatory | | | | |
| drugs) | | | | |
| Aspirin | 17.106 (46.68) | 25.134 (36.32) | 79,843 (11.13) | <0.001 |
| Systemic | 8.405 (22.94) | 12.770 (18.45) | 76,695 (10.69) | <0.001 |
| corticosteroids | | | | |
| DMARDs | 509 (1.39) | 752 (1.09) | 6,628 (0.92) | <0.001 |
| (disease | | | | |
| modifying anti- | | | | |
| rheumatic | | | | |
| drugs) | | | | |
| Systemic | 98 (0.27) | 177 (0.26) | 1,214 (0.17) | <0.001 |
| immuno- | | | | |
| suppresants | | | | |

TABLE 2

| | Effect estimate | Confounder | Disease risk |
|---|---|---|---|
| | matched on age- | adjusted | score adjusted |
| | group, gender, | effect estimate | effect estimate |
| | and year (RR, | (RR, 95% | (RR, 95% |
| Type of | 95% confidence | confidence | confidence |
| CCB Use | interval) | interval) | interval) |
| Current use | 0.77 (0.65, | 0.67 (0.54, | 0.68 (0.58, |
| of any CCB | 0.90)*** | 0.79)*** | 0.78)*** |
| Dihydropyridine | 0.68 (0.54, | 0.63 (0.55, | 0.63 (0.53, |
| CCB group | 0.84)*** | 0.78)*** | 0.79)*** |
| Non- | 0.99 (0.78, | 0.85 (0.75, | 0.73 (0.57, |
| dihydropyridine | 1.27) | 0.93)* | 0.94)* |
| CCB group | | | |

*P < 0.05,
**P < 0.01,
***P < 0.001

TABLE 3

| | | | |
|---|---|---|---|
| | IR % (case/ | DRS-adjusted RR | Trend |
| Category | person-years) | (95% Cl) | P-value |

Cumulative annual duration of CCB use

| | | | |
|---|---|---|---|
| 7-30 days | 1.45% (361/24,805) | Reference | <0.0001 |
| (reference) | | | |
| 31-90 days | 1.40% (308/21,966) | 0.94 (0.80-1.11) | |
| 91-365 days | 0.92% (492/53,317) | 0.63 (0.54-0.72)*** | |

Average daily dose of amlodipine use

| | | | |
|---|---|---|---|
| Non-user | 0.99% (8152/820,610) | Reference | 0.44 |
| Nifedipine 10 | 0% (0/694) | NA | |
| mg | | | |
| Nifedipine 20 | 0.34% (6/1,755) | 0.32 (0.14-0.70)*** | |
| mg | | | |
| Nifedipine 30 | 0.40% (6/1,499) | 0.38 (0.17-0.84)*** | |
| mg | | | |

***P < 0.001

TABLE 4

| | Effect estimate | Confounder | Disease Risk |
|---|---|---|---|
| | matched on age | adjusted | Score adjusted |
| | group, gender, | effect estimate | effect estimate |
| | and year (RR, | (RR, 95% | (RR, 95% |
| | 95% confidence | confidence | confidence |
| Users | interval) | interval) | interval) |
| Use of CCBs | 0.77(0.65, | 0.67(0.54, | 0.68(0.58, |
| vs Nonuse | 0.90)*** | 0.79)*** | 0.78)*** |
| Use of beta- | 0.86 (0.78- | 0.92 (0.81- | 0.99 (0.83- |
| blockers vs | 0.95)** | 1.06) | 1.12) |
| Non-use | | | |
| Use of loop | 1.21 (0.91- | 0.91 (0.79- | 0.88 (0.62- |
| diuretics vs | 1.62) | 1.06) | 1.26) |
| Non-use | | | |

**P < 0.01,
***P < 0.001
RR: relative risk;
CCB: calcium channel blocker

What is claimed is:

1. A preventive treatment method of preventing or mitigating the risk of reactivating latent TB in a subject in need of said preventive treatment, comprising:
administering to the patient a calcium channel blocker.

2. The method of claim 1, wherein said calcium channel blocker is a dihydropyridine calcium channel blocker.

3. The method of claim 1, wherein said calcium channel blocker is one previously approved by the FDA.

4. The method of claim 1, wherein said calcium channel blocker is nifedipine.

5. The method of claim 1, wherein said subject is a person in a latent state of TB infection.

6. The method of claim 1, wherein said subject is a person infected with HIV.

7. The method of claim 6, wherein said subject is in a state of latent TB infection.

8. A pharmaceutical composition useful as a prophylactics for preventing or reducing the risk of latent TB reactivation in a subject infected with HIV, said composition comprising:
a calcium channel blocker;
an anti-HIV agent; and
a pharmaceutically acceptable carrier.

9. The pharmaceutical composition of claim 8, wherein said calcium channel blocker and anti-HIV agent is each selected from one previous approved by the FDA.

10. The pharmaceutical composition of claim 8, wherein said calcium channel blocker is nifedipine.

11. A method of developing and optimizing a pharmaceutical composition useful as a prophylactic treatment to prevent or reduce the risk of reactivating latent TB in a subject infected with HIV, said method comprising:
compiling a structural database of calcium channel blockers and ranking the structures by their effectiveness;
compiling a structural database of anti-HIV agents and ranking the structures by their effectiveness;
generating a pharmacophore model for each of calcium channel blocker and anti-HIV agent based on top 10 structures of each; and
using the pharmacophore models to search and identify potential lead compounds in a chemical structural database not including the calcium channel blockers and the anti-HIV agents used to generate the pharmacophores.
